(19) United States
(12) Patent Application Publication
(10) Pub. No.: US 2026/0256537 A1
(43) Pub. Date: Sep. 3, 2026

(54) APPARATUS, METHODS AND SYSTEMS FOR LASER SAFETY INTERLOCK BYPASS FOR CATHETER

(71) Applicant: CANON U.S.A., INC., Melville, NY (US)

(72) Inventor: Daisuke Yamada, Cambridge, MA (US)

(21) Appl. No.: 18/993,268

(22) PCT Filed: Jul. 11, 2023
(86) PCT No.: PCT/US2023/069940

§ 371 Date: Jan. 10, 2025

Related U.S. Application Data
(60) Provisional application No. 63/388,155, filed on Jul. 11, 2022.

(51) Int. Cl. A61B 90/00; A61B 5/00

(52) U.S. Cl. CPC A61B 90/04; A61B 5/0066; A61B 5/0071; A61B 5/6852; A61B 2090/049

(57) ABSTRACT

The present patent application aims to teach imaging apparatus, methods, and systems for providing a laser safety bypass for at least one optical probe in the apparatus, wherein the bypass interface for connection with a bypass, which when connected, disables the laser interlock.

### CROSS REFERENCE TO RELATED PATENT APPLICATIONS

This application claims priority from U.S. Provisional Patent Application No. 63/388,155, filed on Jul. 11, 2022, in the United States Patent and Trademark Office, the disclosure of which is incorporated by reference herein, in its entirety

### FIELD OF THE DISCLOSURE

The present disclosure relates in general to optical imaging apparatus, methods and systems, and more particularly, to a combined fluorescence and optical coherence tomography catheter having a bypass for the interlocking safety system, such that the laser being locked by the safety system may be enacted for various purposes.

### BACKGROUND OF THE DISCLOSURE

Optical coherence tomography (OCT) provides high-resolution, cross-sectional imaging of tissue microstructure in situ and in real-time, while fluorescence imaging enables visualization of molecular processes. The integration of OCT with fluorescence imaging in a single catheter provides the capability to simultaneously obtain co-localized anatomical and molecular information from the subject tissue, such as an artery wall. For example, in “Ex. Vivo catheter-based imaging of coronary atherosclerosis using multimodality OCT and NIRAF excited at 633 nm” (Biomed Opt Express 2015, 6(4): 1363-1375), Wang discloses an OCT-fluorescence imaging system using He:Ne excitation light for fluorescence and swept laser for OCT simultaneously through the optical fiber probe.

In optical imaging systems, a patient interface unit (PIU) is used to interface between the catheter and the system console. The PIU comprises of a fiber optic rotary joint, rotational motor, translational motor and drivers for the motors. In order to acquire cross-sectional images of tubes and cavities such as vessels, esophagus and nasal cavity, the optical probe, which is housed in catheter sheath, is rotated with a fiber optic rotary joint (FORJ) with the rotational motor. In addition, the optical probe is simultaneously translated longitudinally during the rotation with the translational motor so that helical scanning pattern images are obtained. This translation is most commonly performed by pulling the tip of the probe back towards the proximal end and therefore referred to as a pullback. As a byproduct of optical imaging systems, laser beams are used to illuminate samples such as tissues from the catheter. To minimize exposing the laser light to human eye, the laser controller often has an interlock mechanism to shut down the laser unless a certain criteria has been met.

By way of example, the reference is EP 2698105, titled “Laser Interlock System for Medical Use” to Samsung Electronics Co. LTD, teaches an ultrasound data acquisition unit (210) to acquire ultrasound data on a subject, said ultrasound data acquisition unit comprising an ultrasound probe; a light source unit (220) to generate laser light; and a control unit (230) to turn on or off the light source unit in response to the acquired ultrasound data; wherein the control unit is configured to determine whether or not contact between the subject and the probe occurs using the acquired ultrasound data, and turns on or off the light source unit according to said determination.

The determination is characterized in that the control unit includes: an image generator to generate a 2-Dimentional (2D) ultrasound image using the acquired ultrasound data; a profile detector to detect a profile of the subject from the 2D ultrasound image; and a state determiner to compare the detected profile of the subject with profile sample information corresponding to the subject, so as to calculate a profile difference; wherein the state determiner is configured to determine that contact between the subject and the probe occurs if the calculated profile difference is less than a preset threshold value, and wherein the control unit further includes a light source controller to turn on the light source unit if the state determiner determines that contact between the subject and the probe occurs.

However, as advantageous it is to have safety interlock to minimize the laser exposer to users in normal application, the laser is required to be operational in various assembly and repair modes, for example, service mode, module/unit tests/diagnosis, performances tests, and etc. The interlock mechanism makes it impossible to operate the laser in these instances.

In particular, when the laser interlock input signals are used based on other electrical components/mechanical condition (e.g. —one of the example is that the interlock shut off the laser when optical probe is not spinning), it would be beneficial to have a means to operate the laser without the precursor condition being met.

Accordingly, and in view of the above-referenced issues, the present innovation provides apparatus, methods and systems for alleviating shortcomings in the established art.

### SUMMARY

The present patent application aims to teach apparatus, methods, and systems for eliminating or significantly reducing laser exposure in an optical probe.

In one embodiment, the subject disclosure teaches an optical imaging apparatus having an imaging engine having at least one laser source and a laser controller, and a motor unit having at least one motor and motor controller for controlling the motor; wherein the laser controller is configured with a laser interlock for disabling the laser when the motor is not operational; and wherein motor operation is determined by a motor operational signal from the motor controller, such that the imaging apparatus further comprises a bypass interface for connection with a bypass, which when connected, disables the laser interlock.

In other embodiment, the bypass emulates the motor operational signal, or the bypass has a jumper between an active DC voltage pin and a pin for the laser safety interlock.

In other embodiments the bypass may have a jumper between a GND pin.

In further contemplated embodiment, the bypass is in electrical communication with the controller to control the laser.

The subject innovation may further include a probe comprising the at least one laser source.

Additional embodiment may include the optical imaging apparatus further comprising a patient interface unit in communication with the imaging engine and motor unit.

In yet another embodiment, the subject innovation teaches a bypass intended for electronic communication with an imaging apparatus: wherein the imaging apparatus comprises: an imaging engine having at least one laser source and a laser controller; and a motor unit having at least one motor and motor controller for controlling the motor; wherein the laser controller is configured with a laser interlock for disabling the laser when the motor is not operational; and wherein motor operation is determined by a motor operational signal from the motor controller, wherein the imaging apparatus further comprises a bypass interface for connection with the bypass, which when connected, disables the laser interlock.

In another embodiment, the bypass emulates the motor operational signal.

In further embodiments, the bypass has a jumper between an active DC voltage pin and a pin for the laser safety interlock, or alternatively, the bypass has a jumper between a GND pin.

It is further contemplated that the bypass is in electrical communication with the controller to control the laser.

The subject innovation may further include a probe comprising the at least one laser source.

Additional embodiment may include the optical imaging apparatus further comprising a patient interface unit in communication with the imaging engine and motor unit.

### BRIEF DESCRIPTION OF THE DRAWINGS

Further objects, features and advantages of the present disclosure will become apparent from the following detailed description when taken in conjunction with the accompanying figures showing illustrative embodiments of the present invention.

FIG. 1 is a schematic illustration of an exemplary OCT-fluorescence imaging system according to one or more embodiment of the subject innovation.

FIG. 2 is a system overview of an exemplary OCT-fluorescence imaging system according to one or more embodiment of the subject innovation.

FIG. 3 provides a schematic illustration of an exemplary OCT-fluorescence multi-modality imaging system, according to one or more embodiment of the subject innovation.

FIG. 4 is a block diagram depicting an exemplary OCT-fluorescence multi-modality imaging system, according to one or more embodiment of the subject innovation.

FIG. 5 provides a overview of an exemplary free space beam combiner in the PIU, according to one or more embodiment of the subject apparatus, method or system.

FIG. 6 is side view of an exemplary catheter for OCT-fluorescence multi-modality imaging system, according to one or more embodiment of the subject apparatus, method or system.

FIG. 7 provides an exemplary architecture for a laser safety interlock electrical signal block diagram, according to one or more embodiment of the subject apparatus, method or system.

FIG. 8 is a diagram depicting an exemplary imaging engine I/O panel interface, according to one or more embodiment of the subject apparatus, method or system.

FIGS. 9A and 9B are diagrams detailing exemplary imaging engine I/O panel interface with connections, according to one or more embodiment of the subject apparatus, method or system.

FIG. 10 is a diagram depicting an exemplary fluorescence imaging engine mechanical assembly, according to one or more embodiment of the subject apparatus, method or system.

FIGS. 11A and 11B are images of exemplary laser safety I/O imaging engine interface, according to one or more embodiment of the subject apparatus, method or system.

FIG. 12 is a diagram depicting an exemplary imaging engine I/O panel interface with an exemplary bypass tool, according to one or more embodiment of the subject apparatus, method or system.

FIG. 13 is an image of an exemplary laser safety bypass tool, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

The fiber optic catheters and endoscopes have been developed to access to internal organs. For example in the cardiology, OCT (optical coherence tomography), white light back-reflection, NIRS (near infrared spectroscopy) and fluorescence technology have been developed to see structural and/or molecular images of vessels with a catheter. The catheter, which comprises a sheath and an optical probe, is navigated to a coronary artery.

In order to acquire cross-sectional images of tubes and cavities such as vessels, esophagus and nasal cavity, the optical probe is rotated with a fiber optic rotary joint (FORJ). In addition, the optical probe is simultaneously translated longitudinally during the rotation so that helical scanning pattern images are obtained. This translation is most commonly performed by pulling the tip of the probe back towards proximal end and therefore referred to as a pullback.

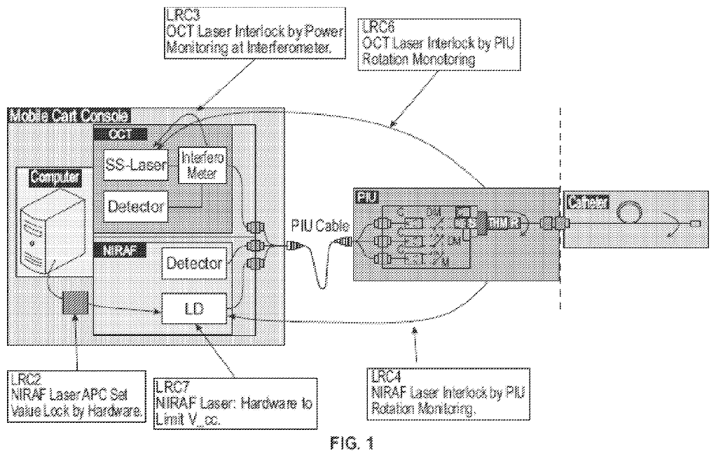

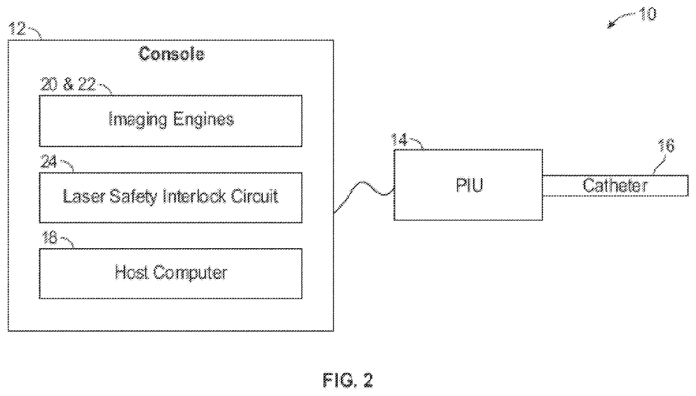

Imaging of coronary arteries by exemplary intravascular OCT and fluorescence systems is described in the 1^{st} embodiment, provided in FIGS. 1 and 2. In this embodiment, the system 10 provides a laser safety interlock to ensure the lasers are off when the optical probe is not spinning.

In detailing the system overview, the imaging system 10 includes a console 12, a PIU 14 (patient interface unit) and a catheter 16. The console 12 comprises a host computer 18, imaging engines (OCT engine 20 and fluorescence engine 22) and laser safety interlock circuits 24. The OCT and fluorescence laser sources 26 and 50 and the controller 62 are housed in the imaging engines 20 & 22, and the laser safety interlock circuits 24 provide the interlock signals to the laser controller 62 to ensure that both lasers 100 (OCT laser and/or Excitation laser) are on only when a rotational motor 58 in the PIU 14 is spinning. The detailed description of each component and their interactions is explained in the following section.

### OCT Engine

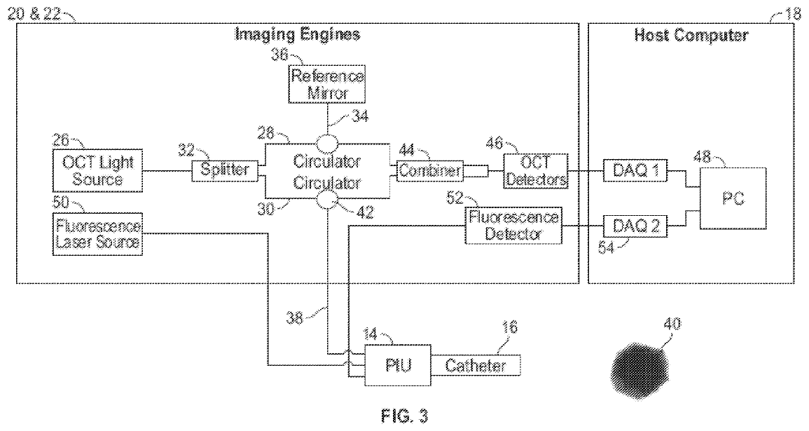

With reference to FIG. 3, an OCT laser beam with a wavelength of around 1.3 um from an OCT light source 26 is delivered and split into a reference arm 28 and a sample arm 30 with a splitter 32. A reference beam 34 is reflected from a reference mirror 36 in the reference arm 28 while a sample beam 38 is reflected and/or scattered from a sample 40 through a PIU 14 (patient interface unit) and a catheter 16 in the sample arm 30. Fibers of the PIU 14 and catheter are made of a DCF (double clad fiber). The OCT laser beam illuminates the sample 40 (outside of the catheter) through the core of DCF, and scattered light from the sample 40 is collected and delivered back to the circulator 42 of an OCT interferometer via the PIU 14 and combined with reference beam 34 at the combiner 44 and generate interference patterns. The output of the interferometer is detected with the OCT detectors 46 such as photodiodes or multi-array cameras. Then signals are transferred to a processor 48 to perform signal processing to generate OCT images. The interference patterns are generated only when the path length of the sample arm matches that of the reference arm to within the coherence length of the light source.

### Fluorescence Engine

An excitation laser with wavelength of 0.635 um from a fluorescence light source 50 delivers to the sample 40 (outside of the catheter) through the PIU 14 and the catheter 16. The patient interface unit (PIU, explained in more detail below) comprises a free space beam combiner so that the excitation light couples into the common DCF with OCT.

The excitation laser 100 illuminates the sample 40 from the distal end of the optical probe in the catheter 16. The sample 40 emits auto-fluorescence with broadband wavelengths of 0.65-0.90 um. The auto-fluorescence is delivered to a fluorescence detector 52 such as photo-multi plier tube (PMT) via the PIU 14. Then, the analog electrical signal at the fluorescence detector 52 is acquired by a data acquisition board (DAQ 2) 54.

### PIU (Patient Interface Unit)

The PIU 14 is interfaced between the catheter 16 and the console 12, and the PIU 14 provides the means to spin and linearly translate the catheter's imaging core (optical probe) within the catheter's outer sheath. The PIU 14 comprises a free space beam combiner, a FORJ 56 (Fiber Optic Rotary Joint), rotational motor 58 and translation motor 60 and linear stage 66, the motor drivers/controllers 62, and a catheter connector 64, as can be seen in FIG. 4.

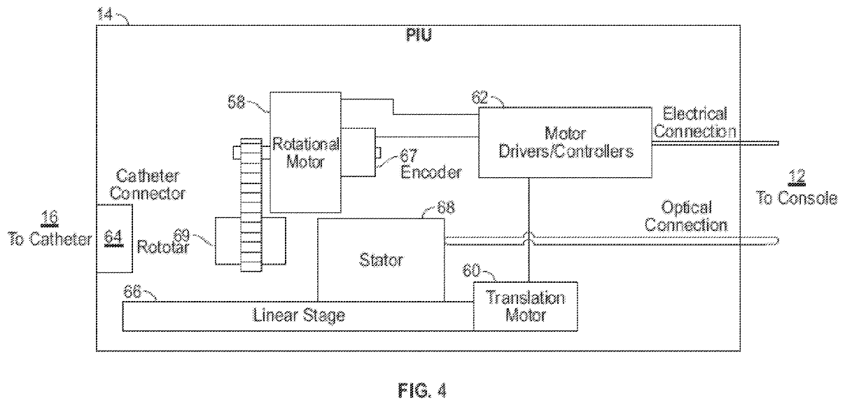

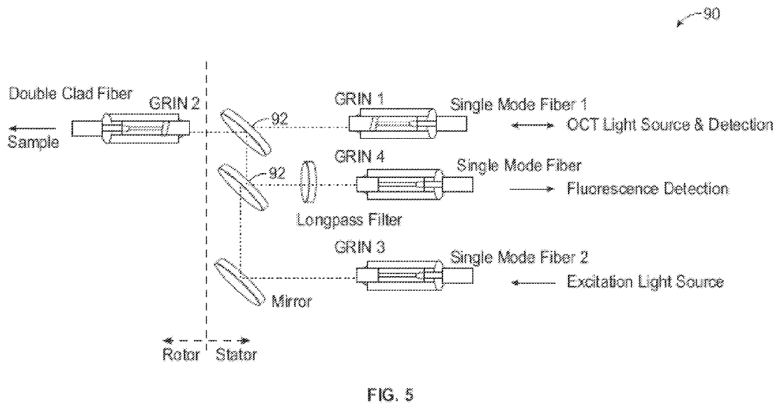

The FORJ 56 (shown in greater detail in FIG. 5) allows uninterrupted transmission of an optical signal while rotating the double clad fiber on the left side along the fiber axis in FIG. 5. The FORJ has a free space optical beam coupler to separate a rotator 69 and a stator 68. The rotator 69 comprises a double clad fiber with a lens to make collimated beam. The rotator 69 is connected to the optical probe, and the stator 68 is connected to the optical sub-systems.

The free space beam combiner 90 has dichroic filters 92 to separate different wavelength lights (OCT laser, excitation laser and auto-fluorescence lights). The beam combiner also comprises low-pass filters or band-pass filters in front of the auto-fluorescence channel to eliminate excitation light to minimize excitation light noises at the fluorescence detector. The cut-of wavelength of the filter (low-pass or band-pass) is selected around from 645 to 700 nm.

The rotational motor 58 delivers the torque to the rotor. Also, the translation motor 60 and linear stage 66 is used for a pullback, and motor drivers/controllers 62 (hereafter referenced as Controller or Motor Driver) drive both rotational motor 58 and translation motor 60. An encoder 67 is attached to the rotational motor 58 to generate encoder signal outputs for feedback to the rotational motor 58 and also to provide the signals to the laser safety interlock circuits 24 to monitor the rotational motor 58 movements.

### Catheter

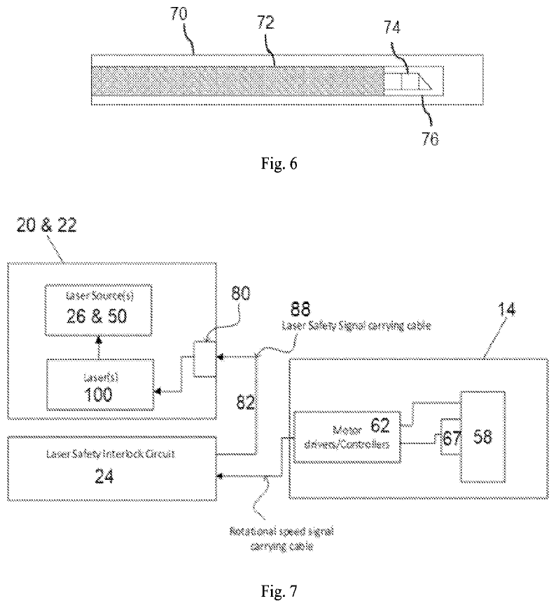

The catheter 16, depicted in FIG. 6, includes a sheath 70, a coil 72, a protector 76 and an optical probe 74. The catheter 16 is connected to the PIU 14. The optical probe 74 comprises an optical fiber connector, an optical fiber and a distal lens 78. The optical fiber connector is used to physically engage with the PIU 14. The optical fiber delivers light to the distal lens 78, and the distal lens 78 is to shape the optical beam and to illuminate light to the sample 40, and to collect light from the sample 40 efficiently.

The coil 72 delivers the torque from the proximal end to the distal end by the rotational motor 58 in the PIU 14. There is a mirror at the distal end of the catheter, so that the light beam is deflected outward. The coil 72 is fixed with the optical probe 74 so that a distal tip of the optical probe 74 also spins to see omnidirectional view of the inner surface of hollow organs such as vessels. The optical probe 74 comprises a fiber connector at proximal end, double clad fiber and a distal lens 78 at distal end. The fiber connector is connected with the PIU 14. The double clad fiber is used to transmit & collect OCT light through the core and to collect auto-fluorescence from samples 40 through the clad. The distal lens 78 is used for focusing and collecting light to and/or from the sample. The scattered light through the clad is relatively higher than that through the core because of the size of the core is much smaller than the clad.

### Laser Safety Interlock

The laser safety interlock circuit 24 provides the laser safety interlock signal 90 to the imaging engine (OCT engine 20 and fluorescence engine 22) via the imaging engines I/O panel 80. The state (HIGH/LOW) of the digital signals 82 are determined based on the rotational speed of the rotational motor 58 in PIU 14, shown in FIG. 7. Wherein the circuit provide a HIGH signals for laser on state, and a LOW signals for laser off state. In this way, this interlock circuitry is fail-safe, in other words, if the laser safety signal losses power for any reason, the laser 100 will default into the off state.

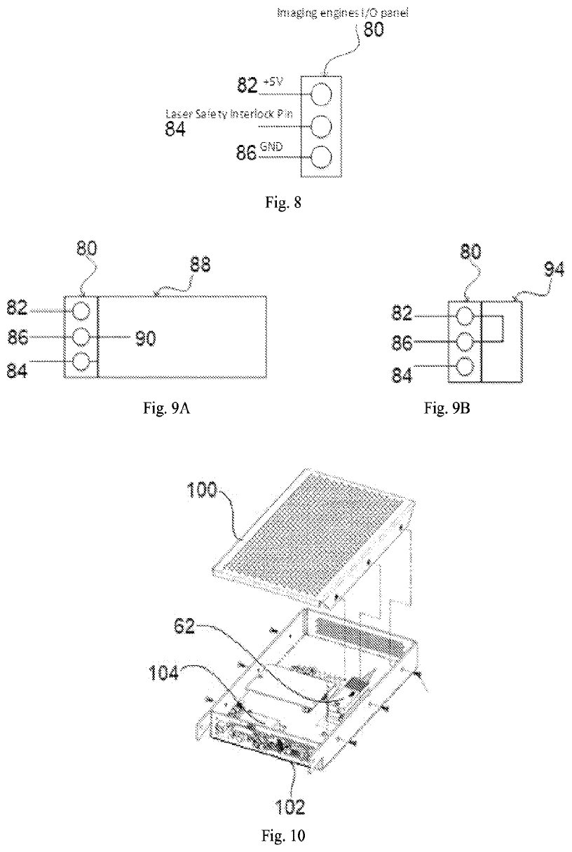

As seen if FIG. 8, the imaging engine 20 & 22 has its own electrical I/O panel 80 to interface with the laser safety interlock signal 90. The electrical I/O panel 80 contains +5V 82, ground 84 (“GND”), and laser safety signal pin 86.

When the laser safety signal carrying cable 88 is connected during normal operation FIG. 9a, the laser safety signal pin 86 is connected to the laser safety interlock signal 90 as well as GND 84 for common ground. The +5V 82 is disconnected during normal operation. The laser safety interlock signal 90 is delivered to the laser drivers/controllers 62, and the laser safety interlock circuit 24 works as intended, as depicted in FIG. 9A.

The subject innovation also teaches a mechanical/electrical laser safety bypass tool 94 to bypass a laser safety interlock 24 without connecting/opening other modules of the system, such that in various modes (e.g. Service—FIG. 9B) the laser may be operational outside the safety interlocks parameters, for instance, in calibrating the lasers. As shown in FIG. 9B, the laser safety interlock 24 would work as intended in normal applications once the bypass tool 94 is removed from the system.

In an alternative embodiment, the system may be adapted for use with imaging of coronary arteries by intravascular OCT and fluorescence system. The system is equivalent to the systems in the 1^{st} embodiment, in that the system provides a laser safety interlock 24 wherein the system allows the fluorescence laser to be on only when the laser safety signals are provided within a set of laser safety parameters.

In this 2^{nd} embodiment, a fluorescence imaging engine 100 contains a laser diode with a wavelength of approximately 635 nm, and a laser driver/controller 62 to drive the laser diode. The imaging engine 100 also provides its own laser safety I/O panel 102 to interface with a laser safety circuitry 104.

At various intervals, the optical power of a laser may need to be calibrated to compensate for optical losses of a system after assembly, as well as during the lifetime of the system. Here, the optical power may be adjusted via the I/O panel 102 interface with the laser safety bypass tool 94. The laser power level is set by a hardware programmable digital potentiometer found in the laser safety bypass tool 94. The potentiometer is programmed by the external USB signals to convert from USB signals to I2C. The software command is allowed only if a laser safety bypass tool 94 is installed.

As detailed in FIGS. 11A and 11B, the I/O panel 102 pinout interface has pinouts of +5V 82, laser safety interlock pin 84, GND 86, Data− 106 and Data+ 108. The Data− 106 and Data+ 108 are used for USB signals. During normal operation, shown in FIG. 11B, the laser safety interlock pin 84 and GND pin 86 are connected to the laser safety circuit to enact the laser safety interlock 24 mechanism.

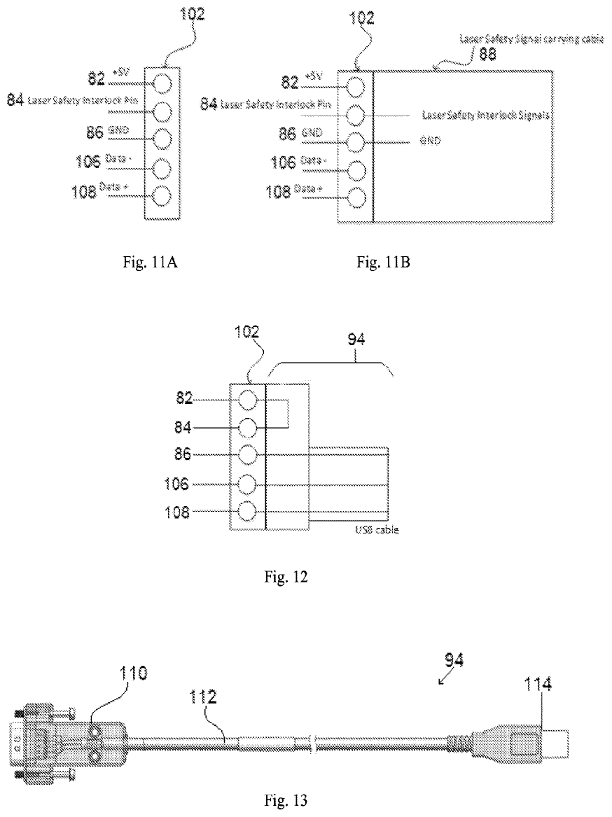

With the laser safety bypass tool 94, shown in FIG. 13, the fluorescence laser optical power is able to be calibrated after assembly without spinning the rotational motor, thus bypassing the laser safety interlock 24. The bypass tool 94 is connected to via the I/O panel pinin interface 110, and the USB cable 112 allows for connection to the calibration apparatus via a USB port 114. The circuitry for this bypass is further shown in FIG. 12, wherein the interlock pin 84 and +5V 82 are connected.

1. Optical imaging apparatus comprising:
an imaging engine having at least one laser source and a laser controller; and
a motor unit having at least one motor and motor controller for controlling the motor;
wherein the laser controller is configured with a laser interlock for disabling the laser when the motor is not operational; and
wherein motor operation is determined by a motor operation signal from the motor controller,
wherein the imaging apparatus further comprises a bypass interface for connection with a bypass, which when connected, disables the laser interlock.

2. The apparatus of claim 1, wherein the bypass enables the motor operation signal regardless of motor operation.

3. The apparatus of claim 1, wherein the bypass has a jumper between an active DC voltage pin and a pin for the laser safety interlock.

4. The apparatus of claim 1, wherein the bypass has a jumper between a GND pin.

5. The apparatus of claims 1, wherein the bypass is in electrical communication with the controller to control the laser.

6. The apparatus of claim 1, further comprising a probe comprising the at least one laser source.

7. The apparatus of claim 1, further comprising a patient interface unit in communication with the imaging engine and motor unit.

8. A bypass intended for electronic communication with an imaging apparatus:
wherein the imaging apparatus comprises:
an imaging engine having at least one laser source and a laser controller; and
a motor unit having at least one motor and motor controller for controlling the motor;
wherein the laser controller is configured with a laser interlock for disabling the laser when the motor is not operational; and
wherein motor operation is determined by a motor operational signal from the motor controller,
wherein the imaging apparatus further comprises a bypass interface for connection with the bypass, which when connected, disables the laser interlock.

9. The apparatus of claim 8, wherein the bypass enables the motor operation signal regardless of motor operation.

10. The apparatus of claim 8, wherein the bypass has a jumper between an active DC voltage pin and a pin for the laser safety interlock.

11. The apparatus of claim 8, wherein the bypass has a jumper between a GND pin.

12. The apparatus of claims 8, wherein the bypass is in electrical communication with the controller to control the laser.

13. The apparatus of claim 8, further comprising a probe comprising the at least one laser source.

14. The apparatus of claim 8, further comprising a patient interface unit in communication with the imaging engine and motor unit.
